(19) United States
(12) Patent Application Publication
(10) Pub. No.: US 2004/0210141 A1
(43) Pub. Date: Oct. 21, 2004

(54) Apparatus and method for dissipating heat produced by TEE probes

(76) Inventor: David G. Miller, North Andover, MA (US)

Correspondence Address: PHILIPS INTELLECTUAL PROPERTY & STANDARDS, P.O. BOX 3001, BRIARCLIFF MANOR, NY, 10510, US

(21) Appl. No.: 10/764,951

(22) Filed: Jan. 26, 2004

Related U.S. Application Data
(60) Provisional application No. 60/463,098, filed on Apr. 15, 2003.

(51) Int. Cl. A61B 1/00; A61B 8/14

(52) U.S. Cl. 600/459; 600/101

(57) ABSTRACT

An apparatus and method are provided for passively dissipating thermal energy produced by Transesophageal Echocardiogram (TEE) probe scanning tips. The apparatus is fabricated from a high thermal conductive material such as Alumina-based ceramics, etc., for placing at the TEE probe tip. The apparatus is placed at a distal end of the TEE probe for dissipating heat produced during a Transesophageal Echocardiography procedure.

The present invention relates generally to medical devices. More particularly, the present invention relates to apparatus and method for dispersing and dissipating heat produced by ultrasonic transducers used in Transesophageal Echocardiogram (TEE) probes.

The heart is one organ for which ultrasonic diagnosis has always been difficult. This is because the heart is located in the thoracic cavity, surrounded by the ribs and lungs. Ultrasonic scanning through the ribs is not a viable option due to the absorptive and reflective characteristics of bone structure. Accordingly, the accepted clinical procedure is to scan the heart intercostally. But the transmission and reception of ultrasound through the intercostal windows is sometimes not clinically useful, because of acoustic reflections from normal body structures, such as the cartilage connected to the ribs.

The advent of endoscopic technology whereby medical devices can be introduced into the body and manipulated external to the body, led to the development of a new technique for ultrasonically scanning the heart-transesophageal echocardiography. By this technique an ultrasonic transducer is located at the end of an elongated probe, which is passed through the patient's mouth and into the esophagus or stomach. From such a position within the thoracic cavity, the ribs no longer pose an impediment to the transmission and reception of ultrasound. The typical transesophageal echocardiogram (TEE) probe includes a control mechanism external to the body, enabling the clinician to manipulate the end of the probe so that the transducer on the probe end is directed as desired toward the heart. This technique, which places the ultrasonic transducer in close proximity to the heart itself, has been found to be most effective in the diagnosis of disease conditions of the heart.

TEE probes used currently in medical examinations are susceptible to overheating. TEE probes are often limited by the thermal rise of the probe surface from transducer self-heating during normal operation. Overheating can also occur due to poor contact between the probe and the patient. Further, future TEE probes will have active circuitry that will add more thermal energy to the tip.

It is common practice to avoid prolonged exposure of the patient to probe tip temperatures in excess of 43° C. in order to minimize the risk of esophageal burns in adult patients. Neonate and pediatric patients, however, may be more vulnerable to these burns than adults; therefore, in these examinations it is recommended to minimize patient exposure time to temperatures in excess of 41° C. To aid in reducing the risk of these burns, many TEE probes employ temperature monitoring systems with warning indicators and auto shutoff features to allow the transducer tip to cool down to a more normal operating temperature. However, such methods also prolong the length of the examination.

The primary object of the present invention is to provide an apparatus and means for reducing the thermal rate of increase at the distal tip of a TEE probe with the use of passive heat dissipation instead of active cooling methods.

This invention provides an apparatus and method for reducing the temperature rise of the probe surface without the use of active cooling methods by making a TEE probe tip thermally conductive in order to dissipate heat.

The present invention addresses two aspects of the overheating phenomenon. The first cause of overheating is due to the inefficient conduction of heat from the transducer to the patient. The second cause of overheating is improper use resulting in poor contact between the probe's acoustic radiating surface and the patient. Both cases result in the sensor overheating and triggering an overheating warning to the operator.

Electrical isolation requirements require that a TEE probe tip be covered with parts that are electrically insulating. Typically, these insulating parts are 0.025″ thick and made of plastic. Plastics commonly used as TEE probe tip covers, however, are poor conductors of heat with a Thermal Conductance (k) of about 0.2 W/M-° K versus a k of about 30 W/M-° K for alumina-based ceramics. Replacing the presently used plastic shell that covers the TEE probe tip with a ceramic shell can reduce temperature rise. Active circuitry which is envisioned for future TEE probes will add additional heat. A shell using a material with high thermal conductivity in accordance with the present invention can readily dissipate this heat.

For a better understanding of the invention, reference is made to the following description of preferred embodiments thereof, and to the accompanying drawings, wherein:

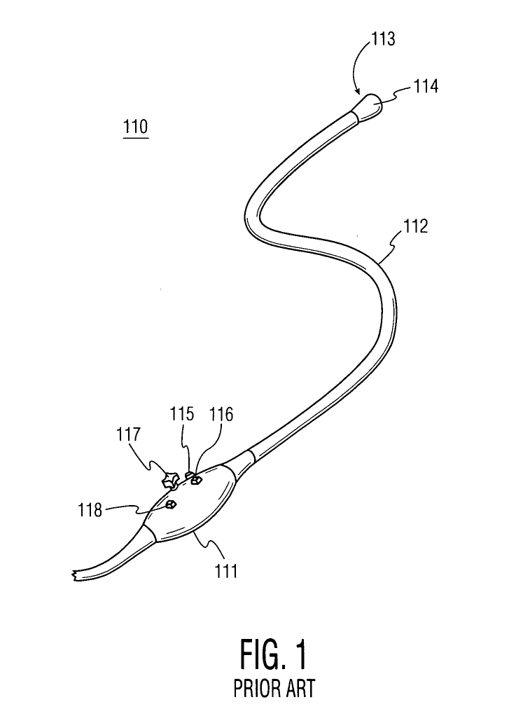

FIG. 1 is a perspective view of a prior art TEE probe;

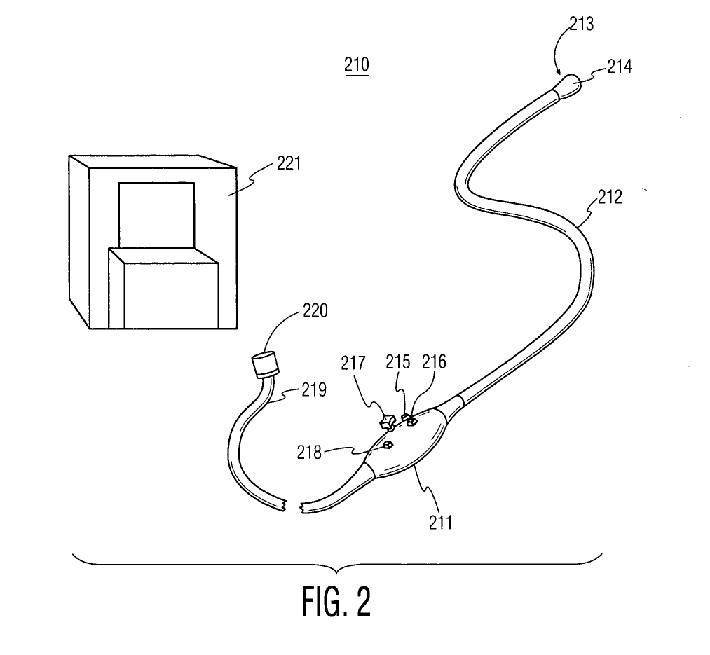

FIG. 2 is a perspective view of a TEE probe in accordance with the present invention;

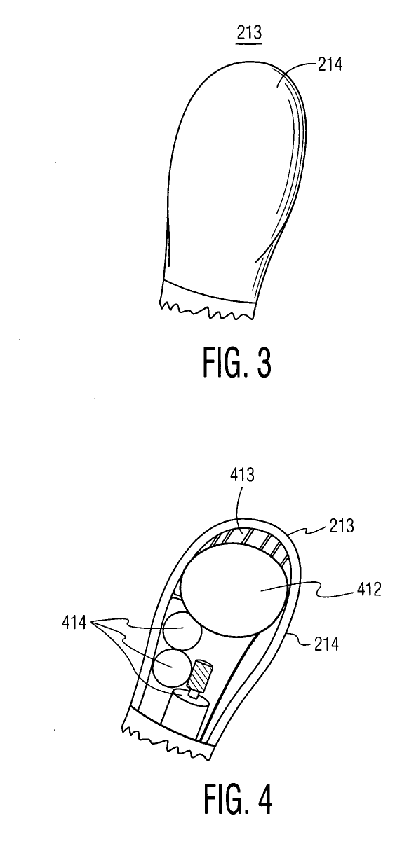

FIG. 3 is an enlarged perspective view of the tip of the TEE probe shown by FIG. 2;

FIG. 4 is a phantom view of the TEE probe tip shown by FIG. 3;

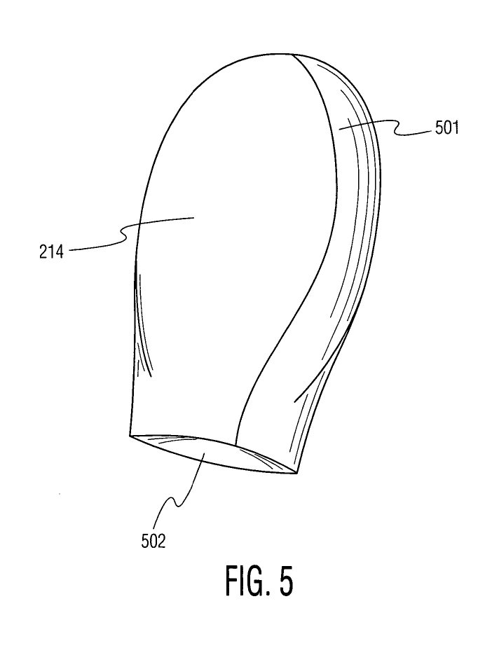

FIG. 5 is a perspective view of a shell for a TEE probe in accordance with the present invention; and

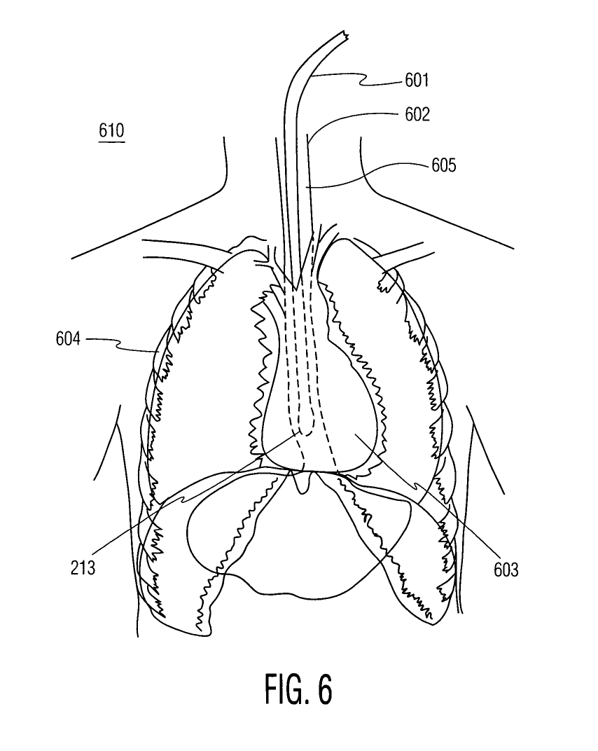

FIG. 6 illustrates the TEE probe shown by FIG. 2 being used in an endoscopic procedure.

The present invention provides an assembly and method for reducing the temperature rise of the probe surface without the use of active cooling methods by making the TEE probe tip thermally conductive. Common causes of overheating are the inefficient conduction of heat from the transducer to the patient and the improper use resulting in poor contact between the TEE probe's acoustic radiating surface and the patient. Both result in the sensor overheating and triggering an overheating warning to the operator. Commonly used transducer lens materials have a low thermal conductivity which causes heat buildup in the vicinity of the transducer. Additionally, the active circuitry that will be employed by future probes will also add heat to the probe tip, making the efficient dissipation of thermal energy a necessary and critical component of TEE probe design.

Plastics commonly used as TEE probe tip covers to provide electrical insulation are poor conductors of heat with a Thermal Conductance (k) of about 0.2 W/M-° K versus a k of about 30 W/M-° K for alumina-based ceramics. Replacing the presently used plastic shell that covers the TEE probe tip with a ceramic shell, in accordance with the present invention, can reduce temperature rise.

A prior art TEE probes is shown by FIG. 1 and designated generally by reference numeral 110. The probe 110 includes a handle 111 where the major controls of the probe 110 are located. Extending from one end of the handle 111 is a gastroscope tube 112. The gastroscope tube 112 is suitable for insertion into a body cavity such as the esophagus. The tube 112 is approximately 100 cm long. At the end of the gastroscope tube 112 is a distal or probe tip 113 where an ultrasonic transducer (not shown) is located. The probe tip 113 is encased in a protective plastic shell 114, a poor thermal conductor. Additionally, two buttons 115 and 116 which control the clockwise and counter-clockwise rotation of the transducer at the tip 113 are located at the handle 111. The probe tip 113 can be articulated in several directions using articulation control knobs 117 located on the handle 111. A brake button 118 is used to lock and unlock the articulation control in any articulated position.

The present invention, as shown in FIG. 2, is a TEE probe 210 which includes a handle 211 where the major controls of the probe 210 are located as in the prior art probe. Extending from one end of the handle 211 is a gastroscope tube 212. The tube 212 is approximately 100 cm long. At the end of the gastroscope tube 212 is a distal or probe tip 213 where an ultrasonic transducer 412 (see FIG. 4) is located. The probe tip 213 is encased in a protective shell 214 (see FIG. 3) made of a material having high thermal and low electrical conductivity such as ceramic or other such bio-suitable material (see Table 1). This allows for the efficient diffusion of thermal energy away from piezoelectric transducer 412 (see FIG. 4) and consequently lowers the overall operating temperature of the probe tip 213.

TABLE 1
Thermal Conductivity of Various Materials

| Material | Thermal Conductivity | Electrical Conductivity |
|---|---|---|
| Plastic | 0.2 W/M-° K. | <10⁻¹³ |
| Ceramic (Alumina) | 30 W/M-° K. | <10⁻¹³ |
| Diamond-Coated Cu | 394 W/M-° K. | <10⁻¹³ |
| Aluminum Nitride | 180 W/M-° K. | <10⁻¹³ |

Extending from the other end of the handle 211 is a cable 219 which terminates at a connector 220. The connector 220 is suitable for connecting the probe 210 to an ultrasound system 221 which energizes the probe 210 and displays images formed from the acoustic signals transmitted and received by the transducer.

Several of the probe controls are also shown in FIG. 2. Two buttons 215 and 216 control the clockwise and counter-clockwise rotation of the transducer at the tip 213 of the probe 210. The probe tip 213 can be articulated in any of several, and preferably four, directions from the handle 211 by articulation control knobs 217. A brake button 218 is used to lock and unlock the articulation control in any articulated position.

As shown in FIG. 4, the transducer 412 and other heat-producing elements 414, i.e. motors, ICs, active circuit elements, etc., within the probe tip 213 radiate their thermal energy to the shell 214 by means of thermally conducting structures 413, liquids, or through direct contact of the heat-producing elements with the outer shell 214 of the probe tip 213.

The material used in the fabrication of the shell 214 is preferably thermally conducting, electrically insulating, non-toxic when used internally on a patient, substantially non-reactive in the presence of bodily fluids, easily disinfected, structurally sturdy, and economically feasible.

The shell 214 of the present invention as shown in FIG. 5 is dimensioned and configured for installation on many commonly used TEE probes i.e. as an operator-installable upgrade. In such a case, the shell 214, made of a high thermal conductive material, is fashioned into various shapes and sizes (OEM part) to accommodate the range of TEE probe tips found in the marketplace. The overall shape and dimensions of the shell 214 is determined by the encased transducer 412 and other components 414, and limited by the need to fit into a patient's esophagus without undo stress to the patient. The shell 214 has a central cavity 502 designed to accommodate the various components of the probe tip and a seam 501 along which the two halves of the shell 214 are joined.

Along the seam 501, tabs, clips or other such structures are employed to securely fasten the two halves while still allowing them to be disconnected when the shell 214 needs replacing. The advantage of this ‘snap together’ design affords a snugger fit with the probe tip, allowing better contact between shell 214 and heat producing components 412 and 414, thus achieving a more efficient heat exchange than can be achieved with a slip-on design. A plurality of shells 214 can be packaged and distributed in various multi-pack packages. Allowing the tip shells 214 to be replaceable affords the operator the benefit of a factory sterilized and undamaged tip for each transesophageal echocardiography procedure.

It is also contemplated for each shell 214 to be a factory installable option, e.g., installable at the time of the manufacture of the TEE probes. The shell 214 can be affixed to the distal tip 213 of the probe 210 either permanently or as an operator replaceable part. The permanently affixed option typically allows for a more integrated fit with the heat generating internal components 412 and 414, such that the heat dissipation is more efficient. The efficiency is derived from better thermal contact being made between the internal components 412 and 414 and the shell 214. However, since a permanently mounted shell 214 cannot be easily replaced, damage to the shell 214 surface such as gouges or cracks can render the entire probe 210 or at least the distal tip 213, in the case of detachable distal tip probe models, unusable.

Referring to FIG. 6, the TEE probe 601 is designed to image the heart 603 without being obscured by reflections and absorptions by the ribs 604. The inventive TEE probe 601 is inserted, into the patient, orally. The probe 601 is then guided through the esophagus 602 to a point adjacent to and behind the heart 603. Care must be taken not to lacerate the esophagus wall 605.

Once the probe tip 213 has been guided to the appropriate position, diagnostic scanning is initiated by the clinician. In order for the TEE probe 210 to produce usable imaging of the heart, and properly dissipate heat buildup at the distal tip, the probe tip 213 must be in contact with the patient 610 at the esophagus wall 605. However, as discussed previously prolonged contact at temperatures above 43° C. in adult patients or 41° C. in pediatric patients can result in burns. The inventive TEE probe lessens this risk by more uniformly and efficiently dissipating this heat buildup thus preventing prolonged overheating of the distal tip components. Consequently, this allows clinicians to complete a scan examination in less time because there are less delays related to probe shutoff caused by overheating and additionally avoids burn-related complications for the patient 610.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

1. An endoscopic imaging apparatus comprising:
an endoscope including a distal end;
at least one ultrasound transducer contained within said distal end; and
a covering fabricated from an electrically insulating material having a Thermal Conductance greater than 1 W/M-° K overlaying at least a portion of said distal end.

2. The endoscopic imaging apparatus as in claim 1, further comprising:
controls for controlling the movement of the distal end;
a signal processor for processing received signals from said at least one ultrasound transducer; and
means for energizing the at least one ultrasonic transducer.

3. The apparatus as in claim 1, wherein said covering is in thermal contact with the at least one ultrasound transducer.

4. The apparatus as in claim 1, wherein said material is non-toxic.

5. The apparatus as in claim 1, wherein said material is non-reactive in the presence of bodily fluids.

6. The apparatus as in claim 1, wherein said material is selected from the group consisting of ceramic and diamond-coated copper.

7. The apparatus as in claim 6, wherein the ceramic is an Alumina-based ceramic.

8. The apparatus as in claim 1, wherein said material has a Thermal Conductance of approximately 30 W/M-° K.

9. An apparatus for dissipating thermal energy produced by an endoscopic imaging apparatus, wherein the apparatus is configured and dimensioned to mate with a distal end of said imaging apparatus for dissipating thermal energy produced at said distal end, said apparatus fabricated from an electrically insulating material having a Thermal Conductance greater than 1 W/M-° K.

10. The apparatus as in claim 9, wherein said material is selected from the group consisting of ceramic and diamond-coated copper.

11. The apparatus as in claim 10, wherein the ceramic is an Alumina-based ceramic.

12. The apparatus as in claim 9, wherein said material is non-toxic when in contact with a patient's internal structures.

13. The apparatus as in claim 9, wherein said material is non-reactive in the presence of bodily fluids.

14. The apparatus as in claim 9, wherein said material has a Thermal Conductance of approximately 30 W/M-° K.

15. A method for scanning a patient's heart using a TEE probe comprising of the steps of:
providing an endoscope having a distal end having a portion thereof fabricated from an electrically insulating material having a Thermal Conductance greater than 1 W/M-° K; and
guiding the endoscope including a distal end.

16. The method as in claim 15, wherein said material is non-toxic.

17. The method as in claim 15, wherein said material is non-reactive in the presence of bodily fluids.

18. The method as in claim 15, wherein said material is selected from the group consisting of ceramic and diamond-coated Copper.

19. The method as in claim 15, wherein the ceramic is an Alumina-based ceramic.

20. The method as in claim 15, wherein said material has a Thermal Conductance of approximately 30 W/M-° K.

21. A device for passively dissipating thermal energy produced by at least one transducer located at a distal end of an endoscopic imaging apparatus, wherein said device is configured and dimensioned to encase the at least one transducer, said device having at least the following properties:
electrically insulating;
a Thermal Conductance greater than 1 W/M-° K; and
substantially non-reactivity in the presence of bodily fluids.
